(19) United States
(12) Patent Application Publication
(10) Pub. No.: US 2010/0204649 A1
(43) Pub. Date: Aug. 12, 2010

(54) High Pressure Intraosseous Bag and Method

(75) Inventors: Larry J. Miller, Spring Branch, TX (US); David S. Bolleter, San Antonio, TX (US); Robert W. Titkemeyer, San Antonio, TX (US)

Correspondence Address: BAKER BOTTS L.L.P.;PATENT DEPARTMENT, 98 SAN JACINTO BLVD., SUITE 1500, AUSTIN, TX, 78701-4039, US

(73) Assignee: VIDACARE CORPORATION, San Antonio, TX (US)

(21) Appl. No.: 12/596,791

(22) PCT Filed: Apr. 23, 2008
(86) PCT No.: PCT/US2008/061258

§ 371 Date: Apr. 19, 2010

Related U.S. Application Data
(60) Provisional application No. 60/913,680, filed on Apr. 24, 2007.

(51) Int. Cl. A61M 5/14; A61J 1/18; A61J 1/10; A61J 1/16

(52) U.S. Cl. 604/134; 604/404; 604/408; 604/131

(57) ABSTRACT

Fluid communication devices may be provided for use with intraosseous devices. Apparatus and methods may also be provided to communicate fluids with an intraosseous device.

### RELATED APPLICATION

This Application claims the benefit of U. S. Provisional Patent Application Ser. No. 60/913,680 entitled “High Pressure Intraosseous Bag and Method” filed Apr. 24, 2007.

### TECHNICAL FIELD

The present disclosure is related to apparatus and methods which may be used to communicate fluids with a patient's vascular system via an intraosseous device.

### BACKGROUND OF THE DISCLOSURE

Vascular access is often essential to viability of a patient in emergency situations, during transportation to a medical facility and during treatment at the medical facility. Obtaining vascular access may be a significant problem in five to ten percent of patients of all ages and weights in pre-hospital and hospital environments. This equates to approximately six (6) million patients in the U.S. annually. For example patients suffering from conditions such as shock, cardiac arrest, drug overdose, dehydration, diabetic coma, renal failure and altered states of consciousness may have very few (if any) accessible veins.

In a hospital or similar medical facility, central line access is often an alternative to IV access. However, central line access generally takes longer, costs more, may have a higher risk of complications and requires skilled personnel to properly insert the central line. In many hospital environments, nurses and physicians are increasingly turning to intraosseous (IO) access as an alternative to IV access, rather than central lines. In pre-hospital environments, paramedics and other emergency medical service (EMS) providers are often finding that IO access may be quick, safe and effective when IV placement is challenging.

Intraosseous (IO) access to bone and associated bone marrow has been used for other procedures including, but not limited to, obtaining biopsy specimens for analysis and research and also for bone marrow transplantation and/or stem cell research.

### SUMMARY OF THE DISCLOSURE

In accordance with teachings of the present disclosure, apparatus and methods may be provided to facilitate access to a patient's vascular system and to communicate fluids with the vascular system. Intraosseous (IO) devices and techniques incorporating teachings of the present disclosure may communicate various fluids including, but not limited to, drugs and medication with the vascular system. A wide variety of conventional intravenous (IV) fluids may be satisfactorily communicated with a patient's vascular system using such IO devices and techniques. Supporting structures, attachment devices and attachment techniques incorporating teachings of the present disclosure may be used to enhance performance of various types of IO devices including, but not limited to, IO devices used to communicate fluids with the vascular system and/or IO devices used to obtain bone and/or bone marrow samples.

One aspect of the present disclosure may include providing apparatus and methods for communicating high pressure fluid to an intraosseous device disposed in a bone and associated bone marrow. Structures, apparatus and techniques incorporating teachings of the present disclosure may be used with a wide variety of intraosseous devices.

In accordance with one embodiment of the present disclosure, a medical apparatus is disclosed. The medical apparatus may include a container, a connector, and an indicator. The container may include a first and a second compartment. The first compartment may have a configuration and dimensions compatible with a standard intravenous fluid bag. The second compartment my at least partially surround the first compartment and may be capable of holding an internal fluid pressure of at least 1000 mm mercury. The connector may be operable to releasably engage the second compartment with a source of fluid pressure. The indicator may be operable to display a pressure measurement of a fluid within the second compartment.

In accordance with another embodiment of the present disclosure, a medical apparatus is disclosed. The medical apparatus may include a container, a spring, a stop, and a trigger. The container may include a compartment having a configuration and dimensions compatible with a standard intravenous fluid bag. The spring may be disposed to expand into the compartment. The stop may be operable to restrict the expansion of the spring. The trigger may be operable to disable the stop.

In accordance with another embodiment of the present disclosure, a method for providing intraosseous fluid delivery is disclosed. The method may include providing a container including a compartment having a configuration and dimensions compatible with a standard intravenous fluid bag. The method may include inserting a fluid bag into the compartment. The method may include connecting the fluid bag in fluid communication with an intraosseous device disposed at an insertion site in a patient. The method may include activating a force against the fluid bag to increase the fluid pressure.

The present disclosure may provide apparatus and methods to establish vascular access during treatment at a wide variety of acute and chronic conditions at locations and facilities including, but not limited to, accident sites, emergency rooms, battlefields, emergency medical services (EMS) facilities, oncology treatment centers, and chronic disease treatment facilities.

Various teachings of the present disclosure may be used during treatment of animals in a veterinary practice.

### BRIEF DESCRIPTION OF THE DRAWINGS

A more complete and thorough understanding of the present embodiments and advantages thereof may be acquired by referring to the following description taken in conjunction with the accompanying drawings, in which like reference numbers indicate like features, and wherein:

FIG. 1 is a schematic drawing showing an isometric view of a powered driver which may be used to place an intraosseous device at a selected insertion site;

FIG. 2 is a schematic drawing showing a side view of a manual driver which may be used to place an intraosseous device at a selected insertion site;

FIG. 3 is a schematic drawing in section and in elevation with portions broken away showing an exploded view of one example of an intraosseous device;

FIG. 4 is a schematic drawing showing an isometric view of the intraosseous device of FIG. 3 disposed in a container;

FIG. 5A is a drawing showing an isometric view of an intraosseous device including a supporting structure and attachment mechanism installed at an insertion site according to one embodiment of the current disclosure; and

FIG. 5B is a schematic drawing in section taken along line 5B-5B of FIG. 5A showing an intraosseous device inserted into a bone and associated bone marrow along with a supporting structure and attachment mechanism incorporating teachings of the present disclosure;

FIG. 6 is a schematic drawing in section showing one example of a connector assembly which may be used to attach a source of fluid with an intraosseous device in accordance with teachings of the present disclosure;

FIG. 7A is a schematic drawing showing one embodiment of a container incorporating a pressure cuff in accordance with teachings of the present disclosure;

FIG. 7B is a schematic drawing showing one embodiment of a container incorporating a pressure cuff in accordance with teachings of the present disclosure;

FIG. 8A is a schematic drawing showing one embodiment of a container incorporating a manual pump in accordance with teachings of the present disclosure;

FIG. 8B is a schematic drawing showing one embodiment of a container incorporating a canister of compressed gas in accordance with teachings of the present disclosure;

FIG. 8C is a schematic drawing showing one embodiment of a container incorporating a pump in accordance with teachings of the present disclosure;

FIG. 9A is a schematic drawing showing one embodiment of a container incorporating a spring in accordance with teachings of the present disclosure;

FIG. 9B is a schematic drawing showing the embodiment of FIG. 9A after the fluid in the container has been dispensed;

FIG. 9C is a schematic drawing showing one embodiment of a container incorporating a spring in accordance with teachings of the present disclosure;

FIG. 9D is a schematic drawing showing the embodiment of FIG. 9C after the fluid in the container has been dispensed;

FIG. 10A is a schematic drawing showing one embodiment of a pressure indicator for use in accordance with teachings of the present disclosure; and

FIG. 10B is a schematic drawing showing one embodiment of a pressure indicator for use in accordance with teachings of the present disclosure.

### DETAILED DESCRIPTION OF THE DISCLOSURE

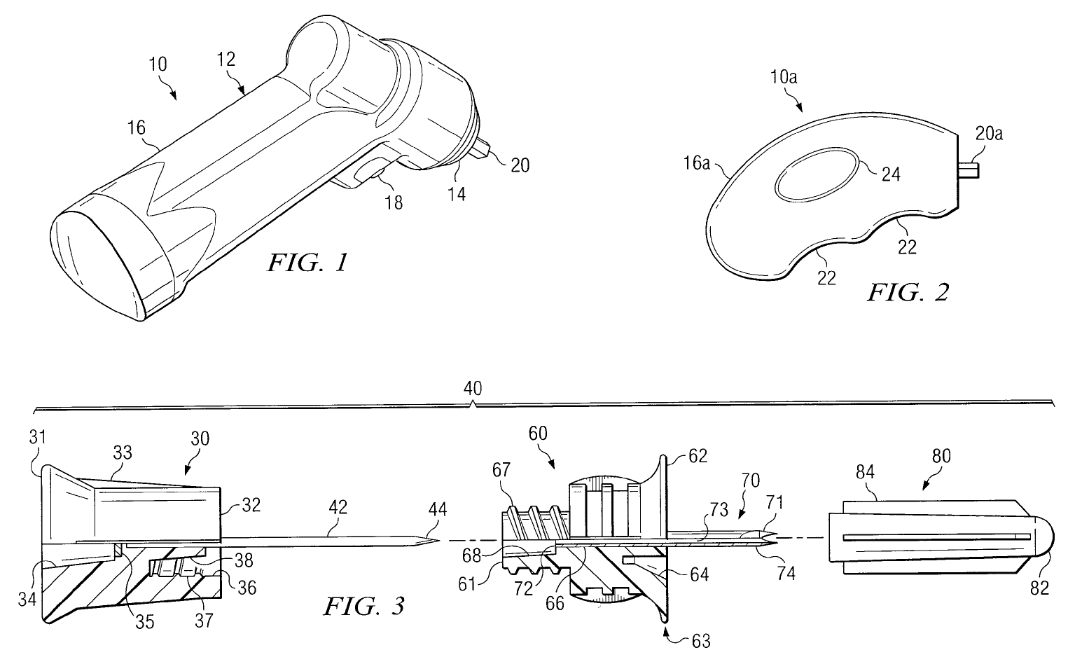

Preferred embodiments of the disclosure and its advantages are best understood by reference to FIGS. 1-10B wherein like numbers refer to same and like parts.

Vascular system access may be essential for treatment of many serious diseases, chronic conditions and acute emergency situations. Yet, many patients experience extreme difficulty obtaining effective treatment because of inability to obtain or maintain intravenous (IV) access. An intraosseous (IO) space provides a direct conduit to a patient's vascular system and systemic circulation. IO access, therefore, is an effective route to administer a wide variety of drugs, other medications and fluids. Rapid IO access offers great promise for almost any serious emergency that requires vascular access to administer life saving drugs, other medications and/or fluids when traditional IV access is difficult or impossible.

The upper tibia proximate a patient's knee or the humeral head proximate a patient's shoulder may be used as insertion sites for an IO device to establish access with the patient's vascular system. Sternal access may also be used as an insertion site. Availability of multiple intraosseous sites has proven to be especially important in applications such as emergency treatment of battlefield casualties or other mass casualty situation. Teachings of the present disclosure may be used at a wide variety of insertion sites.

The distal tibia is located just above the inside of the ankle. This location may more readily provide vascular access to morbidly obese patients. The distal tibia is usually a thinner area of the body. Using the distal tibia as an insertion site may allow emergency medical service personnel to pump medications and fluids into the body of obese patients when regular conventional IV access is difficult. EMS personnel may often not be able to start conventional IV infusions in obese patients because their size may obscure many of the veins used for conventional access. Adipose tissue (fat) around other available IO access sites may be so thick that EMS personnel cannot reach the bone and associated bone marrow with available IO needles. In such cases, disposition of an IO needle in the distal tibia may offer a significant improvement in vascular access to the overweight population.

The humeral head and sternum further provide insertion sites for an intraosseous device located above the diaphragm of a patient. Placing or inserting an intraosseous device above the diaphragm may be preferred by some emergency room physicians and trauma surgeons for rapid vascular access.

Teachings of the present disclosure may be satisfactorily used to communicate fluids with the intraosseous device at a wide variety of locations. For example, apparatus and methods incorporating teachings of the present invention may be used to provide intraosseous access to a patient's vascular system in the sternum, the proximal humerus (the shoulder area), the proximal tibia (below the knee), and the distal tibia (above the inside of the ankle). Teachings of the present disclosure are not, however, limited to IO devices which may be inserted at the tibia, humerus, or sternum.

Intraosseous access may also be used as a “routine” procedure with chronic conditions which substantially reduce or eliminate the availability of conventional IV sites. Examples of such chronic conditions may include, but are not limited to, dialysis patients, seriously ill patients in intensive care units and epilepsy patients. Intraosseous devices along with supporting structure and/or monitoring equipment incorporating teachings of the present disclosure may be quickly and safely used to provide IO access to a patient's vascular system in difficult cases such as status epilepticus to give medical personnel an opportunity to administer crucial medications and/or fluids. Further examples of such acute and chronic conditions are listed near the end of this written description. Insertion sites and associated target areas for IO placement such as a patient's tibia, humerus, or sternum are often larger than insertion sites and associated target areas for placement of an IV device making IO insertion easier than IV insertion.

A person having ordinary skill in the art may recognize that IO fluid delivery is facilitated by delivery of fluid at a higher pressure than that which is typically used in intravenous fluid delivery. Apparatus and methods using teachings of the current disclosure may have application in both IV and IO fluid delivery systems. It is further understood that IO fluid delivery may be facilitated by fluid pressures of up to about 1000 mm Hg, as opposed to typical IV delivery fluid pressures near 300 mm Hg. In fact, some IV fluid delivery may be performed without any external source of pressure relying instead on the effects of gravity to deliver the fluid.

The term “driver” may be used in this application to include any type of powered driver or manual driver satisfactory for installing an intraosseous (IO) device such as a penetrator assembly or an IO needle into a selected target site.

For some applications a powered driver or a manual driver may be directly coupled with an IO device. For other applications various types of connectors may be used to couple a manual driver or a powered driver with an IO device. A wide variety of connectors and associated connector receptacles, fittings and/or other types of connections with various dimensions and configurations may be satisfactorily used to releasably engage an IO device with a powered driver or a manual driver.

The term “intraosseous (IO) device” may be used in this application to include any hollow needle, hollow drill bit, penetrator assembly, bone penetrator, catheter, cannula, trocar, inner penetrator, outer penetrator, IO needle or IO needle set operable to provide access to an intraosseous space or interior portions of a bone. A wide variety of trocars, spindles and/or shafts may be disposed within a cannula during installation at a selected target area. Such trocars, spindles and shafts may also be characterized as inner penetrators. A cannula may be characterized as an outer penetrator.

The term “fluid” may be used within this patent application to include any liquid including, but not limited to, blood, water, saline solutions, IV solutions, plasma or any mixture of liquids, particulate matter, dissolved medication and/or drugs appropriate for injection into bone marrow or other target sites. The term “fluid” may also be used within this patent application to include body fluids such as, but not limited to, blood, bone marrow and cells which may be withdrawn from a target site.

Various features of the present disclosure may be described with respect to powered driver 10 and/or manual driver 10a. Various features of the present disclosure may also be described with respect to intraosseous device 40 and/or hub 60. However, intraosseous fluid delivery systems incorporating teachings of the present disclosure may be satisfactorily used with a wide variety of drivers and intraosseous devices. The present disclosure is not limited to use with intraosseous device 40, hub 60, or drivers 10 or 10a.

FIG. 1 shows an embodiment of a powered driver 10 which may be satisfactorily used to insert an intraosseous device into a selected target area or penetration site. Powered driver 10 may include housing 12 with various types of motors and/or gear assemblies disposed therein (not expressly shown). A rotatable shaft (not expressly shown) may be disposed within housing 12 and connected with a gear assembly (not expressly shown). Various types of fittings, connections, connectors and/or connector receptacles may be provided at one end of the rotatable shaft extending from end 14 of housing 12.

For some applications pin type fitting or connector 20 may be formed on the one end of the rotatable shaft. A matching box type fitting or connector receptacle may be provided on an intraosseous device so that connector 20 of powered driver 10 may be releasably engaged with the intraosseous device. For some applications, with tapered surfaces formed on the exterior thereof.

Handle 16 may include a battery (not expressly shown) or other power source. Handle 16 may also include trigger assembly 18 for use in activating powered driver 10. Examples of powered drivers are shown in pending patent applications Ser. No. 10/449,503 filed May 30, 2003 entitled “Apparatus and Method to Provide Emergency Access To Bone Marrow”, Ser. No. 10/449,476 filed May 30, 2003 entitled “Apparatus and Method to Access Bone Marrow” and Ser. No. 11/042,912 filed Jan. 25, 2005 entitled “Manual Intraosseous Device”.

FIG. 2 shows one example of a manual driver which may be satisfactorily used to insert an intraosseous device into a selected target area. For this embodiment manual driver 10a may be generally described as having handle 16a with a “pistol grip” configuration. Handle 16a has an ergonomic design with finger grips 22 and one or more finger rests 24.

Connector 20a may extend from first end 14a of handle 16a. Connector 20a may have a configuration and dimensions similar to previously described connector 20. However, manual drivers may be provided with a wide variety of connectors and/or connector receptacles. Various details concerning manual drivers are discussed in more detail in pending U.S. patent application, Ser. No. 11/042,912 filed Jan. 12, 2005, entitled “Manual Intraosseous Driver”.

FIG. 3 is a schematic drawing showing an exploded view of one example of a penetrator assembly which may be used to provide access to a patient's vascular system. Penetrator assembly or IO needle set 40 may include connector 30, hub 60 and cover 80. Connector 30 may be described as having a generally cylindrical configuration defined in part by first end 31 and second end 32.

First end 31 may include opening 34 formed with various configurations and/or dimensions. Opening 34 may sometimes be referred to as a “connector receptacle.” For some applications opening 34 may be sized to receive portions of a drive shaft. One or more webs (not expressly shown) may also be formed in first end 31 extending from opening 34. Open segments or void spaces (not expressly shown) may be formed between such webs. Opening 34 and associated webs (if any) may be used to releasably engage connector 30 with either a manual driver or a powered driver.

The configuration and dimensions of opening 34 may be selected to be compatible with releasably engaging connector 30 of IO needle set 40 to connector 20 of powered driver 10 or connector 20a of manual driver 10a. For some applications metallic disk 35 may be disposed within opening 34 for use in releasably engaging IO needle set 40 to a magnet (not expressly shown) disposed on the end of connector 20 or 20a.

For some applications exterior portions of connector 30 may include an enlarged tapered portion adjacent to first end 31. A plurality of longitudinal ridges 33 may also be formed on the exterior of connector 30 proximate first end 31. The enlarged tapered portion and/or longitudinal ridges 33 may allow an operator to grasp associated IO needle set 40 during attachment with a driver and may facilitate disengagement of connector 30 from hub 60 after outer penetrator or cannula 70 has been inserted into a bone and associated bone marrow.

Second opening 36 may be formed in second end 32 of connector 30. The configuration and dimensions of opening 36 may be selected to be compatible with releasably engaging relevant portions of hub 60. For example threads 37 may be formed on interior portions of opening 36 extending from second end 32. Threads 37 may be sized to engage threads 67 formed on an exterior portion of hub 60. In addition, opening 36 may include male luer slip 38, configured to correspond to female luer slip 68 in hub 60. It should be noted that male luer slip 38 and female luer slip 68 do not come into physical contact when connector 30 and hub 60 are connected. Threads 37 and 67 may be characterized as forming portions of a Luer lock connection. However, the present disclosure is not limited to threads 37 and 67. Various types of releasable connections including, but not limited to, other types of locking connections may be formed on adjacent portions of connector 30 and hub 60.

Trocar or inner penetrator 42 may be securely engaged with connector 30 extending from second end 32. The dimensions and configuration of inner penetrator 42 may be selected to allow inner penetrator 42 to be slidably inserted into longitudinal bore 73 of outer penetrator or cannula 70. Trocar 42 may include first end or tip 44. The dimensions and configuration of tip 44 may be selected to accommodate inserting inner penetrator 42 into bone and associated bone marrow at a selected target area in a patient.

Hub 60 may include first end or distal end 61 and second end or proximal end 62. In this context, distal end 61 and proximal end 62 are noted in relation to the patient. First end 61 may include any features selected to be compatible with connector 30. For example first end 61 of hub 60 may have a generally cylindrical pin-type configuration compatible with releasably engaging hub 60 with second end 32 of connector 30. As another example, hub 60 may include threads 67 formed adjacent to first end 61 of hub 60. Threads 67 may be compatible to be releasably engaged with threads 37 formed on interior portions of opening 36 of connector 30.

For some applications first end 61 of hub 60 may be configured to accommodate various connectors and/or to allow access for various methods of fluid delivery (e.g., a luer lock, a syringe, a standard IV connection and/or a needle). For example, first end 61 of hub 60 may include a check valve (not expressly shown), the check valve operable to allow fluid access via engaged luer lock connections and to restrict fluid access in the absence of an engaged luer lock connector. In another example, first end 61 of hub 60 may include a gasket (not expressly shown) operable to allow fluid access when punctured by a needle and to restrict fluid access in the absence of an engaged needle.

For some applications second end 62 of hub 60 may include flange 63. The dimensions and configuration of second end 62 of hub 60 may be varied to accommodate various insertion sites for an IO device. Hub 60 may be formed with a wide variety of flanges or other configurations compatible with contacting a patient's skin adjacent a desired insertion site.

Passageway 66 may extend from first end 61 through hub 60 to second end 62. Portions of passageway 66 extending from second end 62 may have dimensions selected to be compatible with securely engaging exterior portions of outer penetrator or cannula 70 with hub 60. Second end 72 of cannula 70 may be disposed within passageway 66 between first end 61 and second end 62. First end 71 of cannula 70 may extend from second end 62 of hub 60. Portions of passageway 66 extending from first end 61 of hub 60 may have an enlarged inside diameter to accommodate attachment with various types of fluid connectors.

Cannula or outer penetrator 70 may have longitudinal bore 73 extending from first end 71 to second end 72. Exterior dimensions of trocar or inner penetrator 42 are preferably selected to allow inner penetrator 42 be inserted through outer penetrator 70 with first end 44 of inner penetrator 42 generally aligned with first end 71 of outer penetrator 70 after threads 67 have been engaged with threads 37.

Tip 71 of outer penetrator 70 and/or tip 44 of inner penetrator 42 may be operable to penetrate bone and associated bone marrow. The configuration of tips 71 and 44 may be selected to penetrate a bone, bone marrow and other portions of a patient's body with minimum trauma. For some applications tip 44 of inner penetrator 42 may have a generally trapezoid shape with one or more cutting surfaces.

For some applications tips 71 and 44 may be ground together as a single unit during an associated manufacturing process. Providing a matching fit allows respective tips 71 and 44 to act as a single drilling unit to minimize damage as portions of IO needle set 40 are inserted into a bone and associated bone marrow.

Inner penetrator 42 may sometimes include a longitudinal groove (not expressly shown) that runs along one side of inner penetrator 42 to allow bone chips and/or tissue to exit an insertion site as IO needle set 40 is drilled deeper into an associated bone. Outer penetrator 70 and/or inner penetrator 42 may be formed from various materials including, but not limited to, stainless steel, titanium or any other material having suitable strength and durability to penetrate bone and associated bone marrow. The combination of hub 60 with cannula 70 may sometimes be referred to as an “intraosseous needle.” The combination of trocar 42 with cannula 70 may sometimes be referred to as a “penetrator set.”

Second end 62 and particularly flange 63 may be used to stabilize hub 60 after insertion into a selected target area of a patient. Second end 32 of connector 30 may be releasably engaged from first end 61 of hub 60 after insertion of outer penetrator 70 into associated bone marrow. The depth of such insertion may be dependent upon the distance between tip 71 of cannula 70 and second end 62 of hub 60. Various types of tubing and/or conduit may then be engaged with threads 67 formed on the exterior of hub 60 proximate first end or pin end 61.

Annular slot or groove 64 may be formed within second end 62 and sized to receive one end of protective cover or needle cap 80. Slot or groove 64 may be used to releasably engage cover 80 with hub 60. For some applications cover 80 may be described as a generally hollow tube having rounded end or closed end 82. Cover 80 may be disposed within annular groove 64 to protect portions of outer penetrator 70 and inner penetrator 42 prior to attachment with a manual driver or a powered driver. Cover 80 may include a plurality of longitudinal ridges 84 formed on the exterior thereof. Longitudinal ridges 84 may cooperate with each other to allow installing and removing cover or needle cap 80 without contaminating portions of an associated penetrator needle or IO device. Cover 80 may be formed from various types of plastics and/or metals.

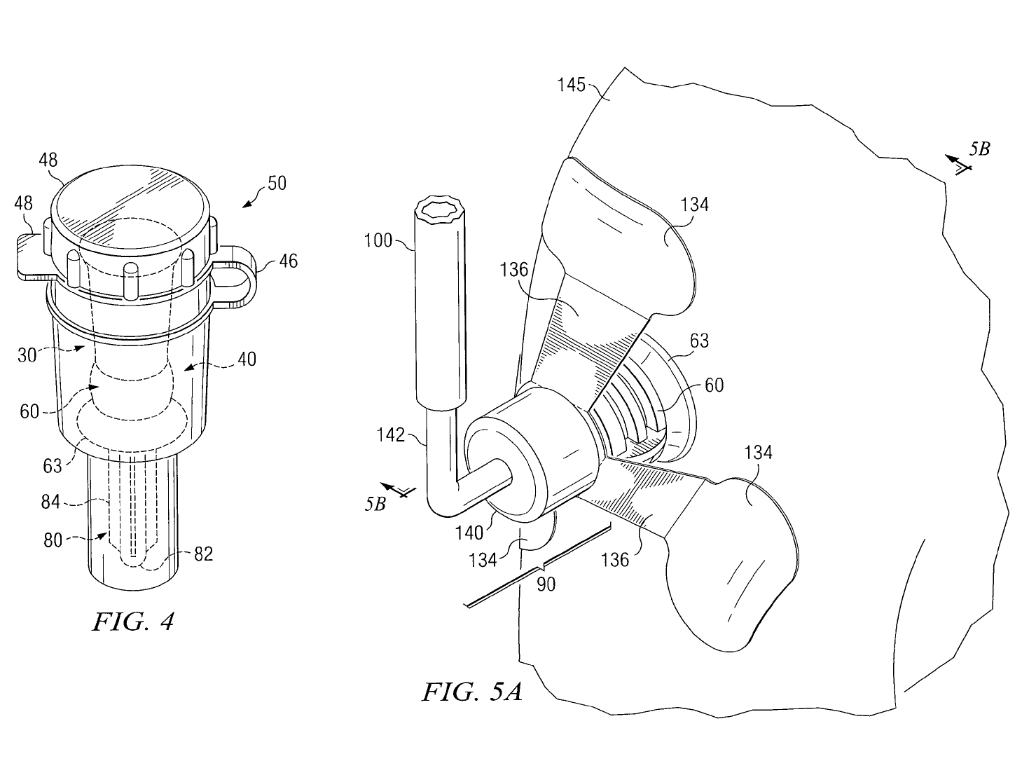

Canister 50 as shown in FIG. 4 may include lid 48. Lid 48 may be configured to allow lid 48 to be flipped open with one or more digits of an operator's hand. With lid 48 open, an operator may releasably engage a driver with an IO device disposed in container. For example, connector 20 of powered driver 10 may be releasably engaged with connector receptacle 34 of connector 30. Flexible connector 46 may be used to retain lid 48 with canister 50 after lid 48 has been opened.

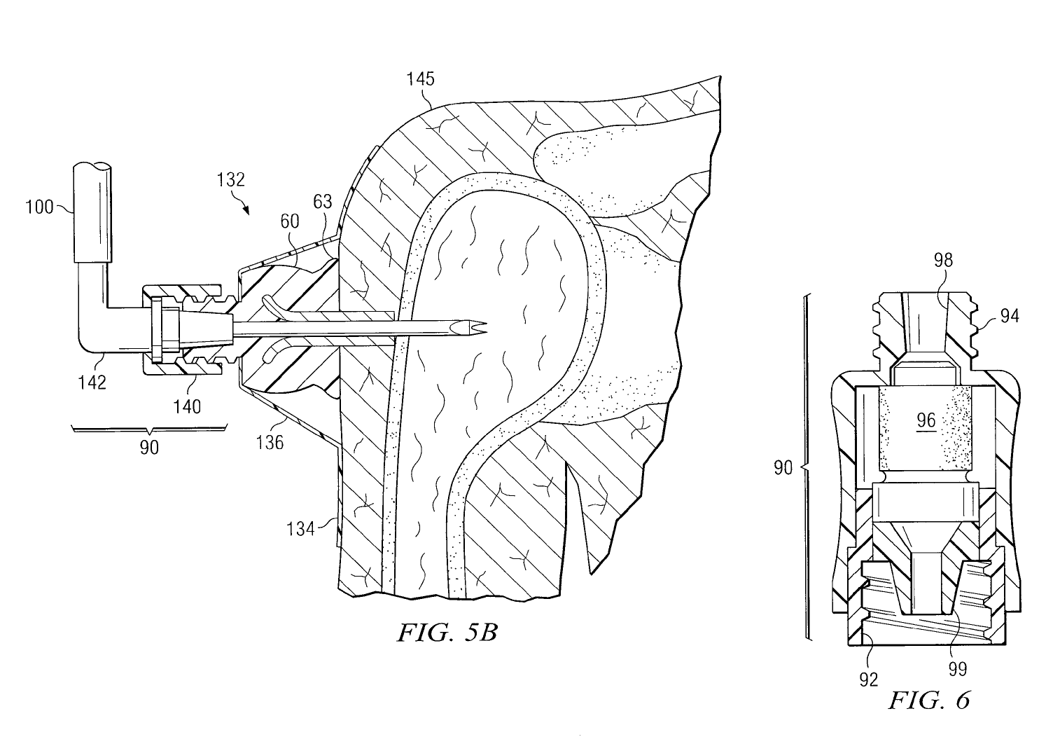

FIGS. 5A and 5B show an intraosseous device inserted into bone and associated bone marrow along with an attachment mechanism and a support structure incorporating teachings of the present disclosure. Various features of the present disclosure may also be discussed with respect to bone 148 and associated bone marrow 146 as shown in FIGS. 5A and 5B. Bone 148 and bone marrow 146 may be representative of a portion of a patient's upper arm or humeral head, but the teachings of the present disclosure are applicable to any suitable bone or bone marrow.

FIG. 5A shows an isometric view of one embodiment of an intraosseous device located in the humeral end of a patient and stabilized with a support structure. In this embodiment, support structure 132 may include wings 136 and three tabs 134, tabs 134 including adhesive layers 138. Adhesive layers 138 may be disposed against a patient's skin 145 in position to provide stability to hub 60. Wings 136 and tabs 134 may be formed from flexible material operable to conform with exterior portions of hub 60 and/or the configuration of an insertion site.

FIG. 5A also shows connector assembly 90 may include any system or device configured to mate with hub 60 and complete a fluid network with the interior of hub 60. For instance, connector assembly 90 may include luer lock cap 140, right angle connector 142, and flexible tubing 100. In some embodiments, right angle connector 142 may comprise any hollow component configured to complete a fluid network between the interior of hub 60 and an external fluid source and/or receiver such as flexible tubing 100. For instance, right angle connector 142 may include rigid tubing, piping and/or other suitable conduits.

FIG. 5B shows a cross section of the embodiment depicted in FIG. 5A, taken along line 5B-5B. As shown in FIG. 5B, an intraosseous device may be generally described as intraosseous (IO) needle 70 having a hollow, longitudinal bore 73 extending therethrough. First end or tip 71 of IO needle 70 may be designed to drill or cut through bone 148 and penetrate associated bone marrow 146. Tip 71 may be open to allow communication of fluids with bone marrow 146.

FIG. 6 shows connector assembly 90 which may be used to communicate fluids with an intraosseous device in accordance with teachings of the present disclosure.

Connector assembly 90 may include any appropriate features or components selected to be compatible with external features of hub 60 or tubing extending therefrom. In some embodiments, such as that shown in FIG. 5A, connector assembly 90 may include internal threads 92 selected to be compatible with threads 67 disposed on hub 60.

Connector assembly 90 may also include any appropriate features or components selected to facilitate attachment to any suitable connections (e.g., extension tubes) for fluid delivery or monitoring devices. For example, connector assembly 90 may include external threads 94 selected to be compatible with a luer lock or other threaded connection.

Connector assembly 90 may include components intended to allow fluid access to hub 60 when appropriate connectors are present. For example, connector assembly may include plug 96. Plug 96 may be any compressible material (e.g., rubber and/or synthetic rubber). In such embodiments, connector assembly 90 may be configured so that plug 96 is under at least some compression in order to create a liquid seal against an inner surface of connector assembly 90. For example connector assembly 90 may include a Halkey-Roberts luer activated valve. One having ordinary skill in the art may recognize additional available medical equipment that may be compatible with the IO devices described herein.

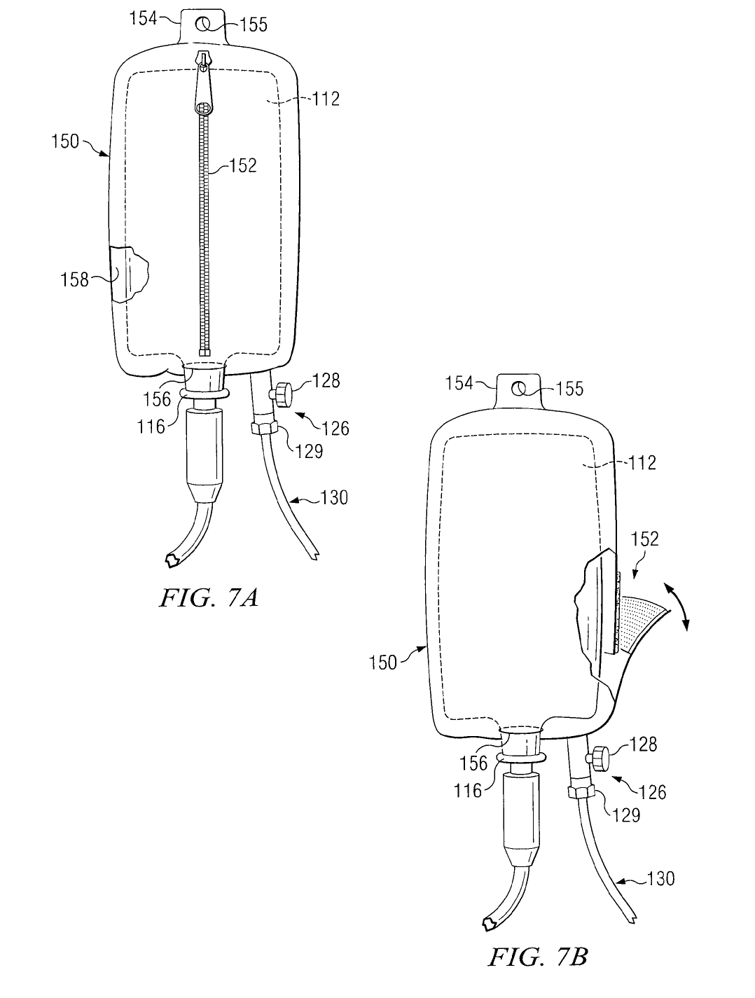

FIGS. 7A and 7B show embodiments of high pressure apparatus operable to pressurize IO fluids in accordance with teachings of the present disclosure. In embodiments such as those shown in FIGS. 7A and 7B, fluid bag 112 may be disposed inside container 150. For some applications container 150 and fluid bag 112 may be formed from generally flexible material.

FIG. 7A shows one embodiment of container 150 in accordance with teachings of the present disclosure. Container 150 may include first compartment 158, fastener 152, hanging tab 154, opening 156, and connector 126.

First compartment 158 may include any feature of container 150 sized to hold fluid bag 112. First compartment 158 may be formed by any suitable process so that fluid bag 112 may be accepted or inserted into first compartment 158. For instance, first compartment 158 may be formed by operation of fastener 152. In an another embodiment, first compartment 158 may be inherent in the construction or design of container 150.

Fastener 152 may include any device operable to releasably secure container 150 around fluid bag 112. For example, as shown in FIG. 7A, fastener 152 may be operable to join opposing sides of container 150 so as to complete formation of first compartment 158. In such embodiments, a user may place fluid bag 112 within container 150 and operate fastener 152 to join container 150 around fluid bag 112. For example, fastener 152 may include zippers, tape, Velcro brand hook and loop fasteners, buckles, clips and/or any other device operable to releasably connect opposing portions of container 150. In other embodiments (not expressly shown), fastener 152 may be operable to releasably fasten fluid bag 112 to container 150 so as to avoid undesired relative motion between fluid bag 112 and container 150.

Fluid bag 112 may include any fluid bag. In some embodiments, fluid bag 112 may include flexible sides so that external physical forces may exert pressure on any fluid disposed within fluid bag 112. For example, fluid bag 112 may be a standard flexible IV bag formed from flexible plastic type material. In alternative embodiments, fluid bag 112 may include other fluid bags such as the VISIV® flexible intravenous container marketed by Hospira, Inc., a specialty pharmaceutical and medication delivery company. In embodiments such as shown in FIG. 7A, fluid bag 112 may include fluid port 116. Fluid port 116 may include any components configured to connect fluid bag 112 to flexible tubing 100 and/or other appropriate fluid conduits for delivery of IO fluid.

Hanging tab 154 may include any feature of container 150 or fluid bag 112 configured so as to facilitate use of the IO fluid delivery system. In some embodiments, such as that shown in FIG. 7A, hanging tab 154 may include an extended tab of container 150 including hole 155. Hole 155 may include any feature of hanging tab 154 configured to releasably connect container 150 to standard medical equipment, such as a feeding/IV pole and/or hooks. In other embodiments, container 150 may be configured to allow use of fluid bag 112 including one or more holes for hanging on an IV pole and/or hooks. In other embodiments, there may be no hanging tab 154 as the teachings of the present disclosure provide fluid delivery that does not depend on gravity or hanging the fluid bag for energy.

Opening 156 may include any device or feature of container 150 configured to allow portions of fluid bag 112 to protrude from container 150. In other embodiments, opening 156 may include any physical opening in container 150 configured to allow operation of connector 116 in relation to flexible tubing 100.

Connector 126 may be any component configured to releasably connect container 150 with a source of fluid pressure. Connector 126 may include valve 128 and fitting 129 with hose or tube 130 extending therefrom. Valve 128 may include any component configured to regulate or restrict the flow of fluid into and/or out of container 150. In addition, valve 128 may include any appropriate actuator, such as a handle and/or an electromechanical actuator (not expressly shown). For example, valve 128 may include a 2-way stop cock, a 3-way stop cock, a one-way valve, and/or any other device configured to regulate the flow of fluid. Fitting 129 may include any component configured to releasably attach container 150 to a source of fluid pressure. Fitting 129 may be configured to attach container 150 to hose 130, a bulb squeezer 118, a carbon dioxide cartridge and/or a hydraulic source of pressure.

FIG. 7B shows another embodiment of container 150 which may include fastener 152, hanging tab 154, and opening 156. Fastener 152 may be operable to releasably connect overlapping portions of container 150. In such embodiments, a user may place container 150 around fluid bag 112 and press opposing faces of fastener 152 to releasably fix container 150 around fluid bag 112.

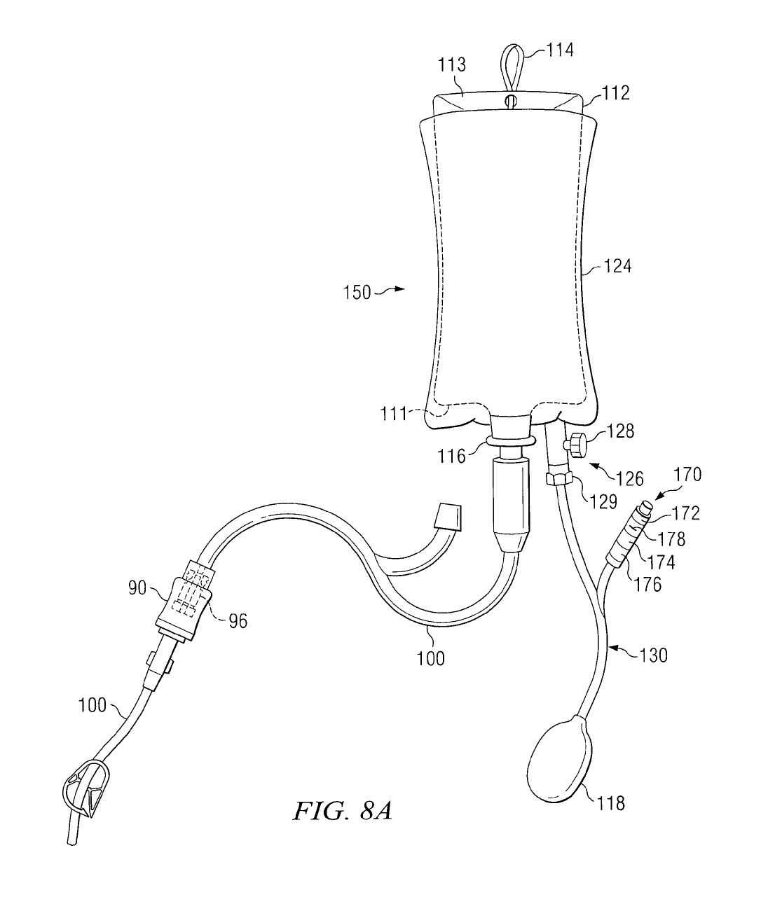

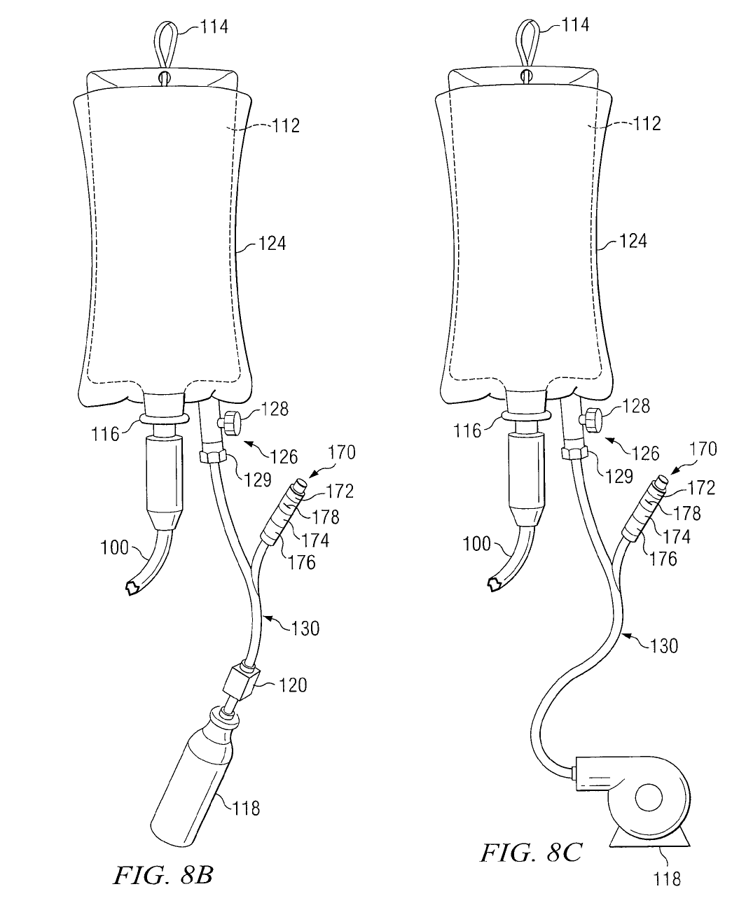

FIGS. 8A-8C show embodiments of container 150 attached to various sources of fluid pressure, in accordance with teachings of the present disclosure. A person having ordinary skill in the art will be able to apply the teachings of this disclosure to many sources of fluid pressure. This disclosure is meant to offer a representative, but not an exclusive list of options.

FIG. 8A shows container 150 and associated flexible tubing 100 operable to connect fluid bag 112 to connector assembly 90 as well as pressure connector 126 and associated tubing and pressure source 118 along with associated pressure gauge 170. For embodiments such as shown in FIG. 8A, container 150 may comprise pressure cuff 124. Fluid bag 112 may be disposed in pressure cuff 124. First end 111 of fluid bag 112 by be disposed proximate fluid port 116. Second end 113 of fluid bag 112 may extend from pressure cuff 124.

Pressure source 118 may be used to control and/or increase the force applied to the fluid in bag 112 by pressure cuff 124. In embodiments such as that shown in FIG. 8A, pressure source 118 may include a manual bulb. In some embodiments, pressure source 118 may include a bulb similar in configuration and operation to the bulb used to inflate a pressure cuff for blood pressure testing. In embodiments including pressure cuff 124 and bulb 118, bulb 118 may be operable to inflate pressure cuff 124 through manual and/or automatic compression.

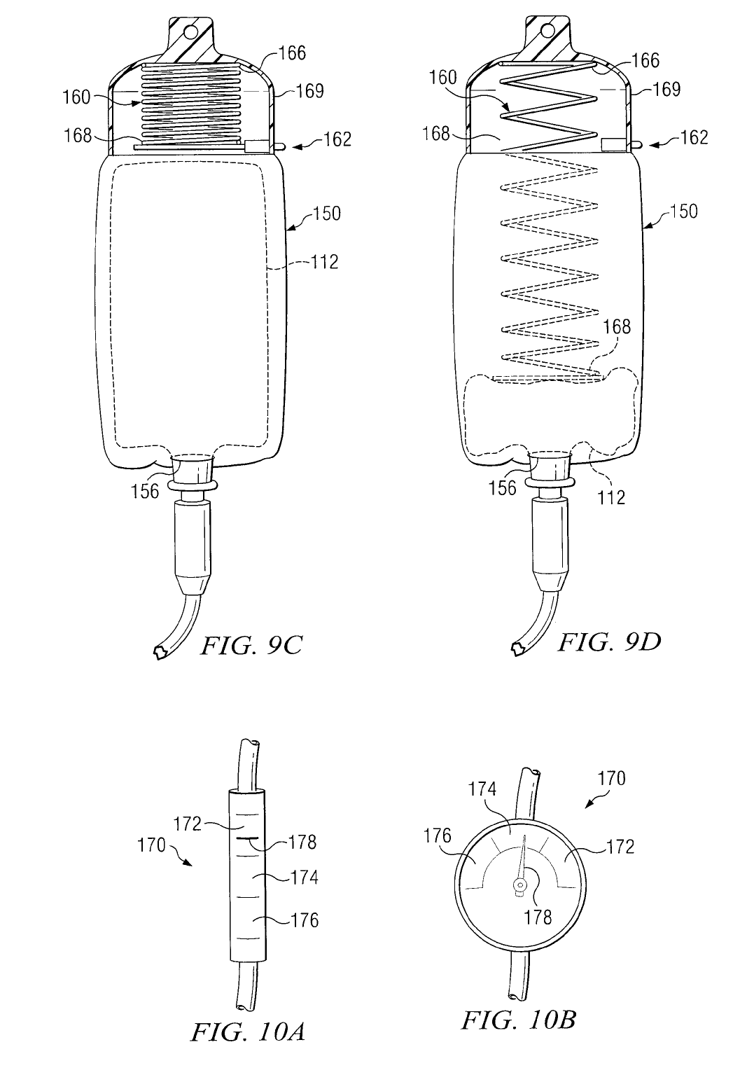

In some embodiments, such as those shown in FIGS. 8A-8C, pressure source 118 may include associated pressure gauge 170. Some embodiments of pressure gauge 170 are discussed in relation to FIGS. 10A and 10B. In any embodiment, pressure source 118 may include a pressure relief valve or other device operable to limit the amount of pressure delivered to pressure cuff 124 or the fluid in bag 112.

Pressure cuff 124 may include extension 114. Extension 114 may include any physical feature configured to extend through a hole included in fluid bag 112 and releasably connect fluid bag 112 to a hook and/or other hanging device. For example, extension 114 may be configured to pass through the hanging hole of a standard flexible IV container and loop over the hook of a standard IV pole (not expressly shown).

Flexible tubing 100 may be any conduit appropriate for communicating fluid from fluid bag 112 to connector assembly 90 and/or IO device 60. In embodiments such as that shown in FIG. 8A, flexible tubing 100 may include surgical tubing and/or medical tubing.

FIG. 8B shows another embodiment of the present disclosure including pressure cuff 124 in which pressure source 118 includes a compressed gas cartridge. In such embodiments, pressure source 118 may contain any high pressure compressed gas available. For example, pressure source 118 may include carbon dioxide cartridges such as those used to power airguns, flat tire repair kits and/or beverage dispenser systems. In other embodiments, pressure source 118 may include compressed gas from any source (e.g., a compressed gas cylinder, an automobile tire, a pneumatic line, a plant compressed air line and/or any other source of compressed gas).

Embodiments including an electrical or mechanical source of pressure may further include an electrical or mechanical device to control the flow rate of fluid in pressure cuff 124 or to control the pressure or flow rate of compressed fluid delivered to pressure cuff 124 by pressure source 118. For example, FIG. 8B shows a flow controller 120 operable to set or limit the pressure of compressed gas delivered by pressure source 118. Flow controller 120 may include any device or apparatus operable to regulate the pressure of a compressed gas or pressurized fluid (e.g., a valve that automatically cuts off the flow of pressurized fluid if it reaches a certain pressure level). Flow controller 120 may operate in association with pressure gauge 170 or with any other feature of container 150.

FIG. 8C shows another embodiment of the present disclosure including pressure cuff 124 in which pressure source 118 includes a pump. In such embodiments, pressure source 118 may include any device for compressing and delivering fluid (e.g., pneumatic and/or hydraulic). For example, pressure source 118 may include a hydraulic pump configured to cooperate with a pre-existing hydraulic system (not expressly shown) to provide high pressure water to pressure cuff 124. In other embodiments, pressure source 118 may include any device configured to provide compressed fluid (e.g., a centrifugal pump, a diaphragm pump, and/or any other sort of pump).

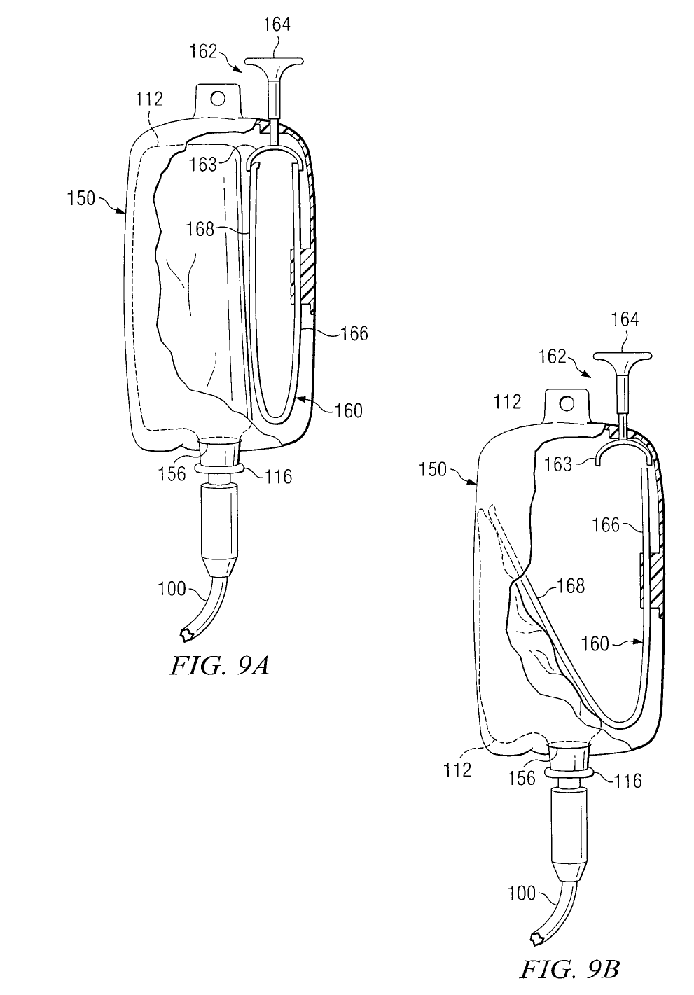

FIGS. 9A-9D show another embodiment of container 150 including compressible mechanism 160 operable to provide physical pressure to fluid bag 112 contained therein. In such embodiments, container 150 may include a spring or similar mechanical device configured to apply physical force to fluid bag 112 when activated.

FIGS. 9A and 9B show two views of one embodiment of container 150 according to the teachings of the present disclosure including opening 156, spring 160, and trigger 162. Spring 160 may include any mechanical device operable to store potential energy through physical compression and operable to release that potential energy through physical expansion. For example, in embodiments such as that shown in FIGS. 9A and 9B, spring 160 may include a leaf spring. As shown in FIG. 9A, spring 160 may store potential energy in its folded state. As shown in FIG. 9B, spring 160 may have transferred that potential energy to fluid bag 112 through physical expansion into its expanded state. Spring 160 may include fixed side 166 and moving side 168.

In embodiments such as that shown in FIGS. 9A and 9B, spring 160 may be provided in a compressed state so that a user may be spared the need to compress the spring. In such cases, spring 160 may be removed from container 150 and replaced with another spring 160 disposed as shown in FIG. 9A. In such embodiments, a kit including container 150 and several springs 160 may be provided for users desiring to reuse container 150 but without means or opportunity to compress spring 160 before each use.

Trigger 162 may include any physical detent operable to restrict the expansion of spring 160 and further operable to release the detent upon operation. For example, as shown in FIG. 9A, trigger 162 may include clamp 163 configured to releasably engage with spring 160. Clamp 163 may be configured to withstand the force exerted by spring 160 when fully compressed. Trigger 162 may include handle 164 configured to be compatible with a user's fingers or hand. Handle 164 may include any device or feature of trigger 162 formed in any suitable manner so as to activate trigger 162 when pulled.

FIG. 9B shows an embodiment in accord with the present disclosure after the fluid stored in fluid bag 112 has been dispensed. Spring 160 may be in an extended state and fixed side 166 may disposed away from moving side 168. Fluid bag 112 may be significantly reduced in volume compared to its initial state.

FIGS. 9C and 9D show two views of one embodiment of container 150 according to the teachings of the present disclosure including opening 156, spring 160, and trigger 162. In embodiments such as that shown in FIGS. 9C and 9D, spring 160 may include a coiled spring. As shown in FIG. 9C, spring 160 may store potential energy in its compressed state. As shown in FIG. 9D, spring 160 may have transferred that potential energy to fluid bag 112 through physical expansion into its expanded state. Spring 160 may include fixed end 166 and moving end 168.

Container 150 may include top 169. Top 169 may be configured to be a removable portion of container 150 operable to compress spring 160 and restrict moving end 168 prior to operation of trigger 162. Top 169 may be provided as a replacement part for use with reusable container 150. In such embodiments, the user may be spared the need to compress spring 160 and, instead, may be able to apply replacement top 169 for the next operation of the device. For example, a kit including container 150 and several tops 169 with preloaded compression of spring 160 may be sold for users desiring to reuse container 150 but without means or opportunity to compress spring 160 before each use. Top 169 may be connected to container 150 through any appropriate components (e.g., threads, clamps, sliding contact, and/or similar devices).

Trigger 162 may include any physical detent operable to restrict the expansion of spring 160 and further operable to release the detent upon operation.

FIG. 9D shows an embodiment in accord with the present disclosure after the fluid stored in fluid bag 112 has been dispensed. Spring 160 may be in an extended state and fixed side 166 may be disposed away from moving side 168. Fluid bag 112 may be significantly reduced in volume compared to its initial state.

Persons having ordinary skill in the art may note that pressure supplied by spring 160 may offer significant advantages to traditional pressure sources relying on the expansion of compressed gasses. For instance, the expansion of compressed gasses typically results in reduced pressure as the volume of the gas increases. In applications relying on a small volume of compressed gas, the effect may result in a notable difference in pressure supplied at the initiation of fluid delivery as opposed to at the end of fluid delivery. On the other hand, in embodiments such as those shown in FIGS. 9A-9D, spring 160 may be designed to apply substantially similar pressure throughout the time in which spring 160 expands and, therefore, throughout the time fluid in fluid bag 112 is delivered.

FIGS. 10A and 10B depict embodiments of pressure indicator 170 for use in practicing the teachings of the present disclosure. Pressure indicator 170 such as those shown in FIGS. 10A and 10B may include demarcations showing ideal operating pressure 172, medium operating pressure 174, and low operating pressure 176 as well as signal 178. In some embodiments, such demarcations may include color coded ranges, text labels or any other suitable indicator. Pressure indicator 170 may be associated with container 150, pressure source 118, or at any other location in which pressure indicator 170 may be operated to measure and report the fluid pressure flowing through pressure indicator 170.

As previously discussed, delivery of fluid through intraosseous transfusion may often require pressures in excess of those traditionally employed for intravenous fluid delivery. Apparatus and methods practicing the teachings of the present disclosure, then, may often require monitoring and control of the pressure applied.

For example, as shown in FIG. 10A, pressure indicator 170 may include a straight segment of conduit including a graduated pressure scale. Portions of the scale may be separated by color bands or bold lines into ideal operating pressure 172, medium operating pressure 174 and low operating pressure 176. Signal 178 may be a moving pointer, a floating bar and/or any other device appropriate for indicating the pressure of fluid flowing through pressure indicator 170.

FIG. 10B shows one embodiment of pressure indicator 170 including signal 178. In such embodiments, pressure indicator 170 may include a dial configured to display operating pressure based on the angular displacement of signal 178. Pressure indicator 170 may include coded segments of a dial indicating ideal operating pressure 172, medium operating pressure 174 and low operating pressure 176.

Although the present disclosure and its advantages have been described in relation to intraosseous devices, it should be clear to a person having ordinary skill in the art that these teachings can be applied to support a variety of medical devices in relation to a patient. For example, embodiments of the present disclosure might be utilized to provide fluid to any intravenous connection or device, a central line, an endotracheal tube, a chest tube, a catheter, dialysis tubing and/or any other device intended to make a fluid connection to one or more systems of the patient.

Examples of acute and chronic conditions which may be treated using intraosseous devices and procedures incorporating teachings of the present disclosure include, but are not limited to, the following:

Anaphylaxis (epinephrine, steroids, antihistamines, fluids, and life support)

Arrhythmia (anti-arrhythmics, electrolyte balance, life support);

Burns (fluid replacement, antibiotics, morphine for pain control);

Cardiac arrest (epinephrine, atropine, amiodarone, calcium, xylocaine, magnesium);

Congestive heart failure (life support, diuretics, morphine, nitroglycerin);

Dehydration (emergency port for life support, antibiotics, blood, electrolytes);

Diabetic Ketoacidosis (life support, electrolyte control, fluid replacement);

Dialysis (emergency port for life support, antibiotics, blood, electrolytes);

Drug overdose (naloxone, life support, electrolyte correction);

Emphysema (life support, beta adrenergics, steroids);

Hemophiliacs (life support, blood, fibrin products, analgesics);

Osteomyelitis (antibiotics directly into the site of infection, analgesics);

Pediatric applications (shock, dehydration, nutrition, electrolyte correction);

Seizures (anti-seizure medications, life support, fluid balance);

Shock (life support fluids, pressor agents, antibiotics, steroids);

Sickle cell crisis (fluid, morphine for pain, blood, antibiotics);

Trauma (emergency port for life support fluids, antibiotics, blood, electrolytes);

Although the present disclosure and its advantages have been described in detail, it should be understood that various changes, substitutions and alternations can be made herein without departing from the spirit and scope of the disclosure as defined by the following claims.

What is claimed is:

1. A medical apparatus comprising:
a container having a first and a second compartment formed therein;
the first compartment having a configuration and dimensions compatible with a standard intravenous fluid bag;
the second compartment at least partially surrounding the first compartment;
the second compartment operable to apply force to the first compartment;
the first compartment capable of holding an internal fluid pressure of at least 1000 mm mercury in response to force applied to the first compartment by the second compartment;
a connector operable to releasably engage the second compartment with a source of fluid pressure; and
an indicator operable to display a pressure measurement of a fluid within the second compartment.

2. The medical apparatus of claim 1 wherein the medical device further comprises:
a fastener operable to releasably connect at least two portions of the container; and
the first compartment at least partially formed by operation of the fastener.

3. The medical apparatus of claim 2 wherein the fastener comprises at least one fastener chosen from the group consisting of a zipper, a hook and loop fastener, a buckle, and a clip.

4. The medical apparatus of claim 1 wherein the container further comprises a blood pressure cuff.

5. The medical apparatus of claim 1 wherein the container further comprises the container having an opening sized to allow fluid connectors to be attached to a fluid bag in the first compartment.

6. The medical apparatus of claim 1 wherein the connector further comprises at least one component chosen from the group including a one-way valve. a two-way valve, and a three way stop cock

7. The medical apparatus of claim 1 further comprising a regulator operable to control the delivery of fluid from the source of fluid pressure to the second compartment.

8. The medical apparatus of claim 7 wherein the regulator operates in association with the indicator to control the pressure of any fluid within the second compartment.

9. The medical apparatus of claim 1 wherein the indicator is operable to display pressures around up to 2000 mm Hg.

10. A medical apparatus comprising:
a container having a compartment formed therein;
the compartment having a configuration and dimensions compatible with holding an intravenous fluid bag;
a spring at least partially disposed within the container and to expand into the compartment;
a stop operable to restrict the expansion of the spring; and
a trigger operable to disable the stop.

11. The medical apparatus of claim 10 wherein the spring comprises a leaf spring.

12. The medical apparatus of claim 10 wherein the spring comprises a spiral spring.

13. The medical apparatus of claim 10 further comprising the spring:
operable to be removed from the container;
operable to be compressed; and
operable to be replaced in the container.

14. The medical apparatus of claim 10 further comprising a removable section configured to contain the spring when compressed.

15. The medical apparatus of claim 10 further comprising the spring configured to provide substantially constant force against a fluid bag in the compartment as the spring expands into the compartment.

16. The medical apparatus of claim 10 further comprising:
a fastener operable to releasably connect at least two portions of the container; and
the first compartment at least partially formed by operation of the fastener.

17. The medical apparatus of claim 10 wherein the fastener comprises at least one fastener chosen from the group consisting of a zipper, a hook and loop fastener, a buckle, and a clip.

18. The medical apparatus of claim 10 wherein the container further comprises the container having an opening sized to allow fluid connectors to be attached to any intravenous fluid bag in the first compartment.

19. A method for providing intraosseous fluid delivery comprising:
providing a container including a compartment having a configuration and dimensions compatible with holding an intravenous fluid bag;
inserting a fluid bag into the compartment;
connecting the fluid bag in fluid communication with an intraosseous device disposed at an insertion site in a patient; and
activating a force against the fluid bag to increase the fluid pressure.

20. The method of claim 19 wherein the force comprises the delivery of high pressure fluid to the container.

21. The method of claim 20 further comprising regulating the delivery of high pressure fluid to pressure around 2000 mm Hg.

22. The method of claim 19 wherein the force comprises the expansion of a spring at least partially disposed within the compartment.
